(19) United States
(12) Patent Application Publication
(10) Pub. No.: US 2011/0178488 A1
(43) Pub. Date: Jul. 21, 2011

(54) Sexual Hygienic Composition

(75) Inventor: Dolhay BaLázs, Budapest (HU)

(73) Assignee: DOLHAY KLINIKA EGESZSEGUGYI KFT., Budapest (HU)

(21) Appl. No.: 13/074,621

(22) Filed: Mar. 29, 2011

Related U.S. Application Data
(63) Continuation of application No. 10/552,321, filed on Oct. 7, 2005, now abandoned.

(30) Foreign Application Priority Data
Apr. 9, 2003 (HU) P 0300932
Mar. 10, 2004 (HU) P 0400576

(51) Int. Cl. A61L 15/20; A61K 35/20; A61K 38/47; A61K 35/74; A61P 31/00; A61P 15/02; B29C 35/16

(52) U.S. Cl. 604/367; 424/535; 424/94.61; 424/93.45; 264/28

(57) ABSTRACT

The invention is a sexual hygienic preparation comprising extract prepared from the milk of at least one equid animal. The preparation may contain medically/therapeutically acceptable additive(s) in specified cases. The preparation is formulated preferably as a feminine cleansing gel, feminine cleansing shampoo, feminine cleansing soap, vaginal suppository, vaginal pill, vaginal cleansing liquid, concentrate, water-soluble solid vaginal cleansing preparation, effervescent tablet, granulate, powder, lyophylized Lactobacillus preparation, spermicide preparation; adsorbent material such as dry or wet wipe, sanitary pad, vaginal suppository, containing sexual hygienic preparation.

### CROSS-REFERENCE TO RELATED APPLICATION

This application is a continuation of co-pending U.S. application Ser. No. 10/552,321, which is National Stage of International Application No. PCT/HU2004/000033, filed Apr. 8, 2004, which published in English and claims priority to Hungarian Application No. P 0300932 filed on Apr. 9, 2003 and Hungarian Application No. P 0400576 filed on Mar. 10, 2004. The entire disclosures of U.S. application Ser. No. 10/552,321, International Application No. PCT/HU2004/000033 and Hungarian Application Nos. P 0300932 and P 0400576 are incorporated herein by reference.

### FIELD

The present invention relates to a sexual hygienic preparation, more particularly a hygienic and/or therapeutic and/or preventive and/or rehabilitative preparation.

### BACKGROUND

Known and widely used types of hygienic, therapeutic, preventive, rehabilitative preparation are feminine cleansing lotions, feminine cleansing gels, feminine cleansing shampoos, feminine cleansing soaps, vaginal suppositories, vaginal pills and vaginal cleansing fluids. These can be made of concentrated liquid or water-soluble solid vaginal cleansing agents, such as effervescent tablets, granulates, or powders. The use of Adsorbent materials such as wet or dry wipes, sanitary pads, vaginal tampons in this area are also known, in addition to other vaginal suppositories, vaginal pills, lyophilized Lactobacillus preparations, lubricants and similar products with spermicidal effects. Many of these products may be considered as both sexual hygienic and therapeutic-preventive preparations. Pharmaceutical preparations used for prevention and curing of sexual diseases, as well as for the follow-up treatment thereof, also fall into this group. Typically, all the mentioned preparations act in direct contact with the genital organs. Since it is practically impossible to distinguish between hygienic, therapeutic-preventive and rehabilitative effects, for the purposes of the present description the above mentioned products are collectively referred to as sexual hygienic preparations.

Natural protection of the vagina is known to be provided by Lactobacilli. Lactobacilli produce lactic acid from glycogen in the vaginal wall and create an acidic medium with a pH of about 3.8. Shifts towards the neutral state inevitably lead to the colonization of facultative pathogenic agents, which latter are almost always present in the vagina.

In addition to the natural deterioration caused by ageing of the genital organs and by various gynaecological diseases, a number of modern products, for instance antibiotics, chemotherapeutics, hormonal contraceptives, intrauterine devices, disinfectants, traditional soaps and shampoos, and the water of swimming pools with basic pH values have side effects that decrease the efficiency of the vaginal protective mechanism. Due to the combined effect of the above mentioned factors, about a half of all patients consult gynaecologists because of vaginitis, or get hospitalized because of the consequences thereof.

A solution for restoring the natural protective mechanism of the vagina is disclosed in Hungarian patent No. 215970. There is disclosed a solid, water-soluble preparation that comprises a selective carbon source for Lactobacilli, most preferably lactose, and a buffer system for producing a pH of 3.5-4. The buffer system may consist of sodium hydrogen carbonate and citric acid. The preparation has been in use in several countries for more ten years.

Known natural therapeutic methods include irrigation with coagulated cow's milk and whey. The document JP 59,053,425 describes the application of whey for the moistening of the vagina and for restoring the self-protective mechanism thereof. Documents U.S. Pat. No. 5,645,830 and U.S. Pat. No. 6,004,551 disclose uro-genital preparations containing skim milk powder and Lactobacilli. The therapeutic experience of the Inventor indicates that preparations made from cow's milk are not sufficiently efficient.

It is also known that odd-toed ungulates, more particularly equids such as assess or horses, have albumin milk, similarly to humans, whereas ruminants have casein milk. It is also known that the milk of equids is most similar to mother's milk as far as the composition and characteristics thereof are concerned. The application of ass milk or horse milk (in the following collectively referred to as mare milk) for nutritional and cosmetic purposes is known. Mare milk is, however, not applied in sexual hygienic products.

The document WO95/01100 discloses a process during which preservation of ass milk is performed by fast refrigeration and subsequent slow freezing. Preserved ass milk has to be thawed before using it in skin care and beauty products. Because of technological reasons but also for reasons of comfort and convenient handling, frozen ass milk cannot be conveniently applied in sexual hygiene products.

German patent DE 19,922,932 discloses a cosmetic preparation containing ass milk and vinegar. The document IT 264,637 describes the dermatological application of preparations made from the colostrum and/or milk of equids. Sexual hygienic applications are not mentioned in either of the documents.

Lysozyme, lactoferrin, lactoperoxidase, xanthine-oxidase, vitamin-binding proteins and immunoglobulines are known to be synergic parts of the complex system that protects the organism against pathogens. Lysozyme is an immunostimulant enzyme that also dissolves the cell membrane of Gram-positive microorganisms. Pure lysozyme (E1105) is a registered food preservative, produced in bulk from egg white. Egg white contains about 5,000 ppm lysozyme. Cow milk and mother's milk contain 0.13 and 400 ppm of lysozyme, respectively, with mare milk containing roughly the same amount as mother's milk. The strongest variety of lysozyme is the human-specific one, see Csapó János, Csapónó Kiss Zsuzsa: Tejtermékek a táplálkozásban [Milk products and nutrition]. Budapest: Mezögazda, 2002, p. 348.

According to the document U.S. Pat. No. 2,590,121, lysozyme produced from ass milk, while much more expensive than its widely known counterpart made from egg white, has more intensive bactericidal characteristics. Owing to its higher price, lysozyme produced from ass milk has not become widespread.

The document GB 1,212,704 describes a vaginal suppository that comprises at least one surface-active fatty compound, an effervescent compound or a compound pair, with one of the pharmaceutically active ingredients being lysozyme.

Lactoferrin is the primary iron controlling agent of the living organism. It binds iron from the digestive tract and in body fluids, depriving pathogenic agents from iron and preventing micro-organisms from proliferating. Lactoferrin is present in most body fluids in mammals, including human vaginal mucus, see Cohen, M. S. et al.: Preliminary observation on lactoferrin secretion in human vaginal mucus; Am. J. Obst. & Gyn, v. 157, 1122-25. Lactoferrin is produced on an industrial scale by extracting it from the milk of mammals. Best-known producers of lactoferrin include Morinaga Milk Co., Japan, BioPole, Belgium, and Avonmore-Waterford, USA.

According to U.S. Pat. No. 6,172,040, lactoferrin treatment is applied for the preservation of meat produce. Document U.S. Pat. No. 6,479,627 describes the production of stable water solution of lactoferrin for e.g. ophthalmological solutions. No sexual hygienic applications are mentioned in either document.

### SUMMARY

The main objective of the present invention is to improve the characteristics of sexual hygiene products.

According to the invention, this is achieved by employing extract produced from the milk of at least one equid animal (an animal belonging to the family Equidae), hereinafter also referred to as mare milk. Optionally, the milk extract may be employed with therapeutically/medically acceptable additives. In the present description the term “milk” covers milk obtained in any phase of lactation, beginning with colostrum. The term “extract” refers to chemically non-pure material(s) or mixtures thereof that can be prepared from mare milk by the application of any suitable method. Such methods are particularly suitable which preserve the essential ingredients of mare milk. Particularly preferred method is the obtaining of milk powder on relatively low temperatures, such as by freeze drying. Another preferred method uses whey of mare milk as raw material for the extract. Preparation of milk powder with other known methods is also applicable.

The invention also concerns a sexual hygienic preparation that comprises extract made from the milk of at least one equid animal, in specific cases also comprising therapeutically/medically acceptable additive(s). Further, the invention also relates to the use of mare milk or milk extract for the manufacturing of a sexual hygienic preparation, and to the use of mare milk or milk extract for the purposes of sexual hygienic treatments.

### DETAILED DESCRIPTION

The stratified squamous keratinising epithelium that covers the outer surface of the human body undergoes gradual change along the external pudendum. Surface layers (stratum corneum, stratum lucidum, stratum granulosum) gradually disappear when approaching to the cervical orifice (Orifitium externum uteri). Thus, non-living cover layers are gradually replaced by living cell layers on the pudendum. The covering epithelia on the juxtaposed surfaces of external genitalia adapts to wet and body identical direct contacts. In this area the efficiency of mechanical, osmotic and anti-dehydration protection processes is decreased. This is often the cause for the potential formation in this area of mirror-symmetric purulent ulcers or carcinomas that are informally called “kissing ulcer” or “kissing carcinoma”. This type of mirror-symmetric deformation occurs less frequently on other skin surfaces due to added protection provided by the surface epithelial layers. The covering epithelium of the glandular epithelium also undergoes changes. Merocrine and holocrine glands occur with decreasing frequency as apocrine glands appear. More endoepithelial glands appear, and the products of glandula vestibularis minor and major (Bartholin gland) are secreted into this region. The acidity/basicity of the surface is determined by discharge (mucus) leaking from the vagina. Shifting of the pH of vaginal discharge toward neutral seriously deteriorates the self-protection of the external pudendum. Thus, products designed to cleanse and protect other bodily surfaces may have detrimental effects on the hygiene and protection of intimate areas.

Surprisingly, it has been found that adding to known sexual hygiene ingredients an extract made from the milk of at least one equid animal, the resulting preparation will have an improved efficiency in preventing, curing and in the follow-up treatment of vaginitis and vulvitis. The preparation can help maintain eubacteriosis, that is the bacterial symbiosis that best protects the lower genital tract against pathogenic effects. Using only some selected purified ingredients of the milk did not produce the same results. The exact mechanism of this beneficial effect is not known in detail, but it is believed that the following facts provide some explanation, without restricting the invention to these theories.

The smegma under men's foreskin contains lysozyme that provides natural protection which is lacking in case of circumcised men, who (and also their sexual partners) are less protected against sexually transferred diseases such as those caused by HIV viruses (Parkash et al: Sub-Preputial Wetness—Its Nature; Ann. Nat. Med. Sci. India, 1982, V. 18, 3, 109-112; Lee-Huang S. at al: Lysozyme and RNases as anti-HIV components in beta-core preparations of humanchorionic gonadotropin; Proc. Nat. Acad. Sci. USA, 1999, 96/6, 2678-2681). It was recognised that this natural protection can be substituted by a lysozyme-containing preparation that is applied to the penis or administered into the internal female genital organ.

Because the effect of lysozyme is maximal in a medium having a pH value about 6, this protective effect is not so intensive in the acidic medium (pH about 3.8) of a healthy vagina. This means that lysozyme is more “passive” if administered into the healthy female genital organ. However, in case of any local breakdown of the natural protection provided by lactic acid—which may cause an increase of the pH value of the vagina—the bactericidal and immunostimulant effect of lysozyme contained by the preparation according to the invention will be more dominant precisely when and where it is needed. The bactericidal and immunostimulant effect of lysozyme is described by Parkash et al. and by Lee-Huang S. at al in the literature cited above.

It has also been found that the protective effect of the inventive sexual hygienic preparation is further enhanced if an extract containing lysozyme and/or lactoferrin, produced from the milk of equids and lysozyme and/or lactoferrin of alternative origin are simultaneously contained therein.

The concentration of free amino acids in mare milk is a multiple of the concentration that can be measured in cow's milk. As much as 11-21% of whey proteins of mare milk are immunoglobulines, of which only traces occur in cow's milk (Csapó János, Csapóné Kiss Zsuzsa: Tejtermékek a táplálkozásban [Milk products in nutrition]. Budapest: Mezögazda, 2002). Immunoglobulines contained in mare milk perform the neutralization of toxins produced by bacteria and of toxic proteins released from dead bacteria. Thus, if the above extract is added to sexual hygienic preparations, then the intensity of hyperaemia in the connective tissue of the vaginal wall, potentially leading to congestion, the interstitial oedema and lymphostasis and the reactions of connective tissue producing subjective symptoms can all be successfully decreased or made less intensive.

Traditional industrial milk processing is carried out at high temperatures and pressures, resulting in the loss of several active ingredients present in native milk. Therefore, for the purposes of the present invention it is suggested that the milk is processed at a temperature under 40° C. when obtaining the extract. Preparation of an extract from mare milk on low temperatures is known per se. For example, the company Equi Libre of Nordsvea, Norway, produces mare milk powder with freeze drying (see at www.hoppemelk.no).

It has been found that the ingredients of the extract made from the milk of equids synergically complement the antipathogenic effect of the majority of traditionally used active ingredients. Thereby the self-protective and regenerative abilities of squamous epithelium suffering from hormone deficit or inflammation are significantly increased. Surprisingly, this synergic effect is noticeable already at very low added amounts of ass milk preparation (as low as 0.2 weight percent relative to solid material content).

In a preferred form the extract is milk powder, or powder prepared from acidified milk (sour milk) or whey. The extract may also be a concentrate in a liquid or semi-liquid form. In a preferred embodiment, the mare milk extract forms 0.01-20 weight percent of the solid material content of the preparation according to the invention. Preferred embodiments of the preparation are formulated in various forms of personal care products, otherwise known per se. The inventive composition may be applied to and used with adsorbent products, where the adsorbent material of the products contain the sexual hygienic preparation according to the invention. However, it is common for all products that they ensure or at least facilitate the direct contact of the active ingredients of the preparation with the affected area of the human body. These areas are primarily the genital organs, particularly the vagina. The preparation is also applicable for sexual hygiene products which are used by men.

The following non-limitative examples illustrate potential compositions and testing methods of the preparations according to the invention.

### EXAMPLE

A) 2000 effervescent tablets (M.f.l.a tablettae No MM) were prepared according to a known method from 750 mg sodium hydrogen carbonate, 900 mg lactose, and 10 mg powder made of ass milk. The ass milk powder had been prepared at a temperature below 40° C., using a known method of freeze drying.

B) 2000 effervescent tablets (M.f.l.a tablettae No MM) are prepared from the ingredients of Example 1/A, but without ass milk powder.

Tablets A and B could not be distinguished from each other either by appearance or by dissolution characteristics.

Study 1

All patients taking part in the study, all between 20 and 48 years of age, had been treated with Colpo-Cleaner Iodine. “Colpo-Cleaner iodine effervescent tablet for making solutions for vaginal irrigation” is a registered medical product under Hungarian registration number OGYI T 6107. Patients were divided into two groups and matched according to the cause of their vaginitis, their age and according to their even or odd day of their date of birth. As follow-up treatment, members of both groups applied vaginal irrigation (douche) with freshly prepared, lukewarm fluid. Of 53 patients who returned for controlling consultation, 25 used tablet A and the other 28 patients used tablet B for making the irrigation fluid. Both groups applied Colpo-Cleaner irrigators (produced by Dolhay Kft., Hungary). For the first three days of the study patients irrigated (douched) 3 times a day, while during the following four-day period one irrigation was carried out each day. At the end of this period, patients shared their experiences with a gynaecologist, with the results being evaluated by a third person.

The simultaneous, matched double-blind study resulted in that, while members of the group of 25 experienced the ceasing of discomfort on the average after 4.4 days, the other group experienced the same after 5.9 days on the average.

Study 2

58 women participated in the study. All participants were older than 52years and all had a feeling of vaginal dryness of varying intensity before the study started. The tablets A and B of Example 1 were applied for irrigation once a day with the Colpo-Cleaner irrigator, dissolving before the tablets in lukewarm liquid. Women with odd birthdates used tablets A, while women with even birthdates used tablets B. After 5 weeks±10 days 35 of the original participants returned for control consultation, out of which 18 had been applying tablet A and 17 tablet B. Test evaluation was carried out by a third person, who established that the randomized double-blind study showed detectable difference between the two groups. Of the group using tablet A one person, while of participants of the group using tablet B four persons said that the feeling of vaginal dryness had not become less intensive during the irrigation period. The initial irritation/itching caused by the irrigation liquid has not ceased in the case of three participants using tablet A, while the others mentioned a slight remnant feeling of irritation/itching only after having been asked targeted questions. Of the group using tablet B six women said that the feeling of irritation/itching had not ceased, while to targeted questions six other women responded that a dim feeling of irritation/itching remained.

Studies 1 and 2 showed that the application of tablet A, containing a very small amount of ass milk preparation, lead to better results. Due to the relatively small number of participants, significance tests have not been carried out.

Preparations according to the invention may be produced with compositions different from the ones illustrated in the examples using various known additives and in the form of powder, granulate, or tablets. Liquid or paste-like forms of the preparation can be produced applying liquid extracts. The extract can be made from the milk of ass or some other equid animal. Studies has shown that the difference in the chemical composition of the milk of various horse races and ass is barely discernible, if at all (See Csapó J. et al: A kanca tejének összetétele (The composition of mare's milk); Állattényésztés és Takarmányozás, 1993, 42.2, 131-146, Hungary). What more, not only various horse races, but all equid animals produce milk which is very similar in composition. See for example: Oftedal O T, Jenness R: Interspecies variation in milk composition among horses, zebras and assess (Perissodactyla: Equidae), J Dairy Res. 1988 February;55(1) pp. 57-66. In this study, milk samples of four species of wild equids (onager, Equus hemionus onager; mountain zebra, E. zebra hartmannae; plains zebra, E. burchelli; Przewalski horse, E. caballus przewalskii) and two domesticated equids (ass, E. asinus; pony, E. caballus) were analysed. It was concluded that these closely related species produce milks that are nearly identical in gross composition and that the domestic horse is a representative model for the study of equid lactation. Accordingly, mare milk extract from all types of equid animals should be suitable for the purposes of the present invention.

The mare milk extract content in the various compositions may be different from those specified in the above example. In specific cases medically/therapeutically acceptable additives can also be used. The extract should most preferably contain 0.2-500 mg of mare milk powder per portion. The amount of added extract can be between 0.01-20 weight percent relative to total solid material content. The extract is usually prepared by processing milk below 40° C., for instance by lyophilisation, but the treatment can be carried out at a higher temperature if necessary. The solidified extract can either be milk powder, or for instance, powder prepared from sour milk or whey, where the acidified (sour) milk can be produced by natural enzymatic and/or chemical process, and the powder produced form the sour milk can be prepared from the coarser-dispersion fraction and/or the re-neutralized whey.

The preparation containing the equid milk extract may further contain additional lysozyme and/or lactoferrin. The lysozyme and/or lactoferrin may also have other origin, i.e. it may be prepared from other substance than mare milk. Further additives (primarily as stabiliser) may be 0.01 weight percent pectin and/or 0.1-0.5 weight percent sorbic acid and/or 0.1-0.5 weight percent ethylenediaminetetraacetic acid or their derivatives. The extract may be contained in known adsorbent materials, such as wet or dry wipes, sanitary pads, vaginal tampons, the primary function of which can be advantageously complemented by the extract.

A significant advantage of the preparations according to the invention is that they restore faster the epithelial layers and the acidic coating destroyed by inflammation, so restitutio in integrum can be earlier achieved. It is also advantageous that the inventive preparations prevent the feeling of vaginal dryness felt by menopausal women with greater efficiency. It is believed that these advantageous effects are caused by the active ingredients contained in the extract.

The active ingredients of the extract synergically complement the antipathogenic effect of original agents. The contents of the mare milk extract nourish and protect natural Lactobacilli and also the injured parts of epithelium, and enhance the protective, preventive and therapeutic effect of the preparation, as well as the efficiency of the follow-up treatments.

As an additional advantage, the home use of the inventive vaginal douche preparations is straightforward, simple and efficient.

What is claimed is:

1. A method for making a sexual hygienic preparation, the method comprising the steps of:
obtaining an extract from milk of at least one equid animal at a temperature below 40° C.; and
forming the extract into a sexual hygienic preparation comprising one of a feminine cleansing gel, feminine cleansing shampoo, feminine cleansing soap, vaginal suppository, vaginal pill, vaginal cleansing liquid, concentrate, water-soluble solid vaginal cleansing preparation, effervescent tablet, effervescent powder, granulate, powder, lyophilized Lactobacillus preparation, spermicide preparation, an absorbent wet or dry wiping pad, sanitary pad, and vaginal tampon.

2. The method of claim 1, wherein the step of obtaining the extract is performed by a freeze drying process.

3. The method of claim 1, wherein the step of forming includes adding lysozyme.

4. The method of claim 1, wherein the step of obtaining includes forming the extract as one of milk powder.

5. The method of claim 1, wherein the step of obtaining includes forming extract from sour milk or whey.

6. The method of claim 1, wherein the milk of the at least one equid animal contains lysozyme

7. The method of claim 1, wherein the step of obtaining includes forming the extract as a concentrate in liquid or semi-liquid form.

8. The method of claim 1, wherein the milk of the at least one equid animal contains one or lysozyme, lactoferrin and a combination of lysozyme and lactoferrin.

9. The method of claim 8, wherein the step of forming includes adding one of lysozyme, lactoferrin, and a combination of lysozyme and lactoferrin.

10. The method of claim 1, wherein the step of forming includes forming the preparation with a solid material content comprising 0.01-20 weight percent of the extract.

11. The method of claim 1, wherein the step of forming includes adding 0.1-0.5 weight percent ethylenediaminetetraacetic acid or a derivative thereof and/or 0.1-0.5 weight percent sorbic acid or a derivative thereof and/or 0.01-2 weight percent pectin, to the preparation.

12. The method of claim 1, wherein the preparation comprises the effervescent tablet or effervescent powder and wherein the forming step includes adding a material serving as a selective carbon source for Lactobacilli and a buffer system suitable for maintaining a pH of 3.5-4.3.

13. The method of claim 12, wherein the forming step includes forming the effervescent tablet or effervescent powder as a single dose comprising 2285 mg citric acid, 750 mg sodium hydrogen carbonate, 900 mg lactose and 0.2-500 mg mare milk powder.

14. The method of claim 13, wherein the single dose further comprises one or more therapeutically acceptable additives.

15. A method for making a sexual hygienic preparation, the method comprising the steps of:
providing unpasteurised milk of at least one equid animal;
obtaining an extract from the unpasteurised milk of at least one equid animal; and
forming the extract into one of a powder, granulate, tablet, liquid, semi-liquid, and paste.

16. The method of claim 15, wherein the step of obtaining the extract is performed by a freeze drying process.

17. The method of claim 15, wherein the step of obtaining the extract is performed at a temperature below 40° C.
